(19) United States
(12) Patent Application Publication
(10) Pub. No.: US 2022/0088240 A1
(43) Pub. Date: Mar. 24, 2022

(54) ULTRAVIOLET LIGHT DISINFECTING SYSTEMS

(71) Applicant: W. L. Gore & Associates, Inc., Newark, DE (US)

(72) Inventors: Mark N. Donhowe, Newark, DE (US); John M. Squeri, Newark, DE (US)

(21) Appl. No.: 17/422,280

(22) PCT Filed: Feb. 7, 2020
(86) PCT No.: PCT/US2020/017238

§ 371 Date: Jul. 12, 2021

Related U.S. Application Data
(60) Provisional application No. 62/802,889, filed on Feb. 8, 2019.
(60) Provisional application No. 62/860,599, filed on Jun. 12, 2019.

(51) Int. Cl. A61L 2/10; C02F 1/32

(52) U.S. Cl. CPC A61L 2/10; C02F 1/325; C02F 2303/20; C02F 2201/326; C02F 2303/04; C02F 2201/3228; C02F 2307/14; A61L 2202/11; C02F 2201/3222; A61L 2202/14

(57) ABSTRACT

UV light disinfecting system where UV light is distributed along the walls of a highly reflective tube. In some embodiments, the UV light disinfecting system is flexible. In at least one embodiment, the UV light disinfecting system includes at least one UV-LED positioned external to a highly reflective tube. In exemplary embodiments, the reflective tube includes a plurality of openings that are arranged so as to position each opening adjacent to a corresponding UV-LED such that UV light generated by the corresponding UV-LED is able to pass through the opening and into the reflective tube. The UV light is scattered along the length of the reflective tube to prevent or eliminate the presence of biofilms as well as to disinfect, sterilize, and purify and pathogens within the tube. Methods to mitigate the growth of biofilms in a water conduit is also provided.

The present invention relates generally to ultraviolet disinfection systems, and more specifically, to in-line ultraviolet light disinfection systems for use in applications to reduce or eliminate biofilms and/or to disinfect pathogens within a fluid system.

### BACKGROUND

Biofilms are an association of micro-organisms in which microbial cells adhere to each other on a living or non-living surfaces. Bacterial biofilms are infectious in nature and as such, they represent a considerable hygiene risk in the air, water, food, and health industry. Biofilms may also cause economic losses where an accumulated biomass restricts flow in water piping systems, for example.

Exposure to ultraviolet (UV) light, particularly corresponding to electromagnetic radiation with wavelengths between about 100 nm and about 400 nm, is known to induce degradation to many materials, including biological materials. Exposure to UV light can break down DNA so that a cell cannot reproduce. In addition, UV light can degrade toxins, which makes UV light useful for disinfection or purification purposes. As such, the use of UV light has found applications in disinfecting air, water, food, beverages, and blood components.

Further, UV light can be used in conventional water pipes and pipe systems. However, conventional UV treatment does not provide residual disinfection throughout the plumbing. The UV light will only disinfect where it impinges on the pathogens. Therefore, infection of faucets, showerheads, drains, and pipes may occur in places where UV light exposure does not occur. In the water industry, conventional, well-designed treatment systems locate the UV light source as close to the point of use as possible. However, due to size constraints, conventional UV light disinfection systems typically cannot t be installed directly at the point of use exit. As one example, in a water faucet, a UV disinfection system is typically installed under the counter. Although such a disinfection system may be effective at disinfecting the water flowing through the UV disinfection system, the last few feet of piping after the UV light emitting region (e.g. the faucet tap itself) will not be disinfected. Thus, there is a risk of biofilm accumulation on the pipe and faucet surfaces prior to the water leaving the faucet.

U.S. Pat. No. 9,586,838 discloses an LED-based system for purifying a fluid flowing through a pipe, comprising means for mounting the system on the pipe, a housing, a pliant carrier structure comprising a plurality of LEDs arranged flush with a first surface of the structure and configured to emit radiation in the UV range, wherein when the system is pipe-mounted, the structure is detachably arranged within the housing, and the structure adopts a substantially tubular shape within the housing with the first surface delimiting a purifying chamber, wherein the purifying chamber is in fluid communication with the pipe so that the fluid flowing through the pipe passes, prior to being dispensed, through the purifying chamber where it is exposed to UV radiation of the energized LEDs.

U.S. Publication 2017/0281812 describes approaches for treating a fluid transport conduit with ultraviolet radiation. A light guiding unit, operatively coupled to a set of ultraviolet radiation sources, encloses the fluid transport conduit. The light guiding unit directs ultraviolet radiation emitted from the ultraviolet radiation sources to ultraviolet transparent sections on an outer surface of the fluid transport conduit. The emitted ultraviolet radiation passes through the ultraviolet transparent sections, penetrates the fluid transport conduit and irradiates the internal walls. A control unit adjusts a set of operating parameters of the ultraviolet radiation sources as a function of the removal of contaminants from the internal walls of the fluid transport conduit.

Therefore, there continues to be a need for improved UV treatment systems, particularly for removing biofilms from surfaces.

### SUMMARY

It is an objective of the present invention to mitigate or eliminate the presence of biofilms which may cause infection of faucets, showerheads, drains, and pipes in water systems. It is also an objective to provide a UV light disinfecting system that is flexible and shaped (e.g., tubular) such that it can fit inside tight spaces such as a gooseneck faucet. It is a further objective to provide a UV light disinfecting system that can be operated in two modes: a high power mode to disinfect pathogens while media is flowing through the tubes; and a low power mode to mitigate the growth of biofilms on the wall of the tube.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic illustration of a side cross-sectional view of a UV light disinfecting system in accordance with at least one embodiment;

FIG. 2a is a schematic illustration of an end cross-sectional view of a circular reflector of a UV light disinfecting system in accordance with at least one embodiment;

FIG. 2b is a schematic illustration of an end cross-sectional view of a square reflector of a UV light disinfecting system in accordance with at least one embodiment;

FIG. 3 is a graphical illustration of irradiance vs. distance of the intensity distribution of light inside a UV disinfecting tube in accordance with at least one embodiment;

FIG. 4 is a graphical illustration of a log plot of the intensity distribution of light inside a UV disinfecting tube in accordance with at least one embodiment;

FIG. 5a is a schematic illustration depicting a diagram of a UV disinfecting tube with two LEDs spaced a distance (Δx) from each other in accordance with at least one embodiment;

FIG. 5b is a graphical illustration of a log plot of the intensity distribution of light inside the tube depicted in FIG. 5a for both 90% and 99% diffuse reflectance walls in accordance with at least one embodiment;

FIG. 6a is a schematic illustration depicting the UV light ray paths inside an integrating sphere in accordance with at least one embodiment;

FIG. 6b is a schematic illustration depicting the DNA of a pathogen being impinged with UV light from all angles in accordance with at least one embodiment;

FIG. 7 is a graphical illustration depicting the total diffuse reflectivity from various PTFE materials in accordance with at least one embodiment;

FIG. 8 is a schematic illustration depicting a UV disinfecting tube having an integrated UV LED array in accordance with at least one embodiment;

FIG. 9a is a schematic illustration of a side cross-sectional view of a UV-LED placed in an opening in the reflector wall and which is covered with an encapsulant in accordance with at least one embodiment;

FIG. 9b is a schematic illustration of a side cross-sectional view of a UV-LED placed in an opening in the reflector wall and which is covered with an encapsulant and with a button film in accordance with at least one embodiment;

FIG. 10 is a schematic illustration depicting a side cross-sectional view of a UV-LED placed in an opening in the reflector wall which is covered with an encapsulant and with an inner transparent tube in accordance with at least one embodiment;

FIG. 11 is a schematic illustration of a side cross-sectional view of a UV-LED strip placed on the exterior of an inner transparent tube and an outer reflective tube surrounding the UV-LED strip positioned on the inner transparent tube in accordance with at least one embodiment;

FIG. 12 is a schematic illustration of a side cross-sectional view of a UV light disinfecting system in accordance with at least one embodiment;

FIG. 13 is a schematic illustration depicting the UV light ray paths inside a UV disinfecting tube in accordance with at least one embodiment;

FIG. 14 is a schematic illustration of a side cross-sectional view of UV light disinfecting system and a forming tool in a method of forming a transparent window in accordance with at least one embodiment;

FIG. 15a is a schematic illustration depicting a forming tool for forming transparent windows with a circular cross-section in accordance with at least one embodiment;

FIG. 15b is a schematic illustration depicting a forming tool for forming transparent windows with a rectangular cross-section in accordance with at least one embodiment;

FIG. 16 is a schematic illustration of a side cross-sectional view of a UV light disinfecting system in accordance with at least one embodiment;

FIG. 17 is a schematic illustration depicting a UV disinfecting tube having an integrated UV LED array in accordance with at least one embodiment;

FIG. 18a is a schematic illustration depicting an apparatus used to measure the intensity distribution across a tube diameter in accordance with at least one embodiment;

FIG. 18b depicts the intensity distribution measured according to the apparatus in FIG. 18a which shows a uniform “top hat” distribution profile in accordance with at least one embodiment; and

FIG. 19 is a schematic illustration depicting a side cross-sectional view of a gooseneck faucet housing a UV disinfecting tube in accordance with at least one embodiment.

### DETAILED DESCRIPTION

Persons skilled in the art will readily appreciate that various aspects of the present disclosure can be realized by any number of methods and apparatus configured to perform the intended functions. It should also be noted that the accompanying figures referred to herein are not necessarily drawn to scale, and may be exaggerated to illustrate various aspects of the present disclosure, and in that regard, the figures should not be construed as limiting. Directional references such as “up,” “down,” “top,” “left,” “right,” “front,” and “back,” among others are intended to refer to the orientation as illustrated and described in the figure (or figures) to which the components and directions are referencing.

The present invention provides a UV light disinfecting system where UV light is distributed along the walls of a highly reflective tube to disinfect pathogens in the media flowing through the tube and to mitigate the growth of biofilms on the walls of the tube. Alternately, the UV light disinfecting system is flexible. In at least one embodiment, the UV light disinfecting system includes at least one UV-LED positioned external to a highly reflective tube. In some exemplary embodiments, the reflective tube includes a plurality of openings that are arranged so as to position each opening adjacent to a corresponding UV-LED such that UV light generated by the corresponding UV-LED is able to pass through the opening and into the reflective tube. In other exemplary embodiments, the reflective tube includes a plurality of transparent windows that are arranged so as to position each window adjacent to a corresponding UV-LED such that the UV light generated by the corresponding UV-LED is able to pass through the window and into the reflective tube. The UV light is scattered along the length of the reflective tube to prevent or eliminate the presence of biofilms as well as to disinfect, sterilize, and purify and pathogens within the tube. Methods to mitigate the growth of biofilms in a water conduit is also provided.

Exemplary flexible UV light generation systems include those having a flexible circuit with multiple UV-LEDs. The flexible circuit may include a plurality of conductors, with each UV-LED positioned in independent electrical communication with at least one of the plurality of conductors. It is to be appreciated that the multiple UV-LEDs may be arranged as an array and that the term array, as used herein, may correspond to a spatial distribution of a plurality of objects, such as UV-LEDs and conductors, with one or more of the objects connected to and/or attached to other objects in the array, such as by electrical connections. A UV-LED array may be regular or non-regular, meaning the objects may be uniformly distributed or non-uniformly distributed. An example array may correspond to a ribbon cable, flexible circuit, or flat flexible cable having UV-LEDs attached along various positions of the ribbon cable, flexible circuit, or flat flexible cable.

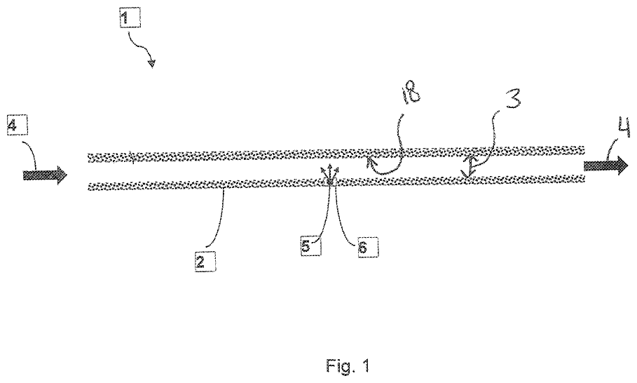

FIG. 1 is a schematic illustration of a side cross-sectional view of a UV disinfecting system in accordance with at least one embodiment. The reflective tube 2 is defined by a tube wall 10 formed of a highly reflective material and an inner diameter 3. The reflective tube 2 has an open interior region. Inner diameters may range from about ⅜ inch to about 2 inches or from about ⅛′ inch″ to greater than 10 inches. In some embodiments, the highly reflective material is largely diffuse reflectance with minimal specular reflectance. Directional arrows 4 indicates the flow of water or air through the highly reflective tube 2.

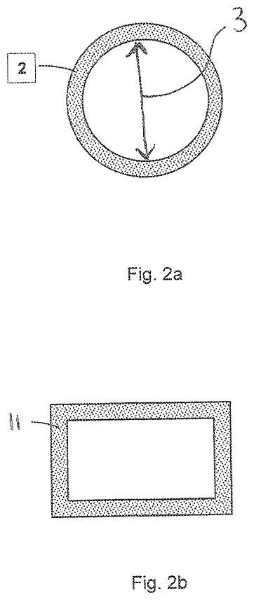

At least one UV-LED 5 is mounted on an external surface of the reflective tube 2 such that UV light emitted from the UV-LED 5 traverses through an opening 6 in the outer wall of the reflective tube 2 and impinges on the inner wall 18 of the reflective tube 2. The UV light then reflects and scatters along the highly reflective tube walls 10, as described in detail below. A cross-section of the UV disinfecting system 1 is shown in FIG. 2a. The tubular system shown in FIG. 1 is generally circular in cross-section. However, it is to be appreciated that the reflective tube 2 is not restricted to circular cylinders, and, in fact, may be formed of any geometric shape. For example, FIG. 2b depicts a rectangular cross-sectional tube 11. It is also to be appreciated that the UV light disinfecting system 1 may not be linear in nature, and may include curves within the highly reflective tube 2, 11. Also, the openings 6 may be formed of a variety of shapes including circles, ovals, triangles, squares, rectangles, diamonds, and other similar shapes. The size of the opening may also vary but is sufficient to allow light from a UV-LED 5 to pass through.

Conventionally, in order to disinfect pathogens flowing through water, a fluency rate on the order of 40 mJ/cm² or 40 mW/sec·cm² is required. It has been determined that by using the UV light disinfecting systems described herein, lower irradiance levels, such as on the order of about 100 nW/cm² and greater, can mitigate or eliminate the growth of biofilms on surfaces, such as the inner surface 10 of the reflective tube 2. It has also been determined that biofilms can be prevented or eliminated by using reflective tubes 2 that contain UV-LEDs that can be left on at all times. For instance, in one embodiment, a high power mode turns on when water flows. When the water is shut off, the UV-LEDs stay on, but at a lower power level. Thus, the UV light is scattered along the inner wall 18 if the highly reflective tube 2 at all times. Switching between the two modes of operation (i.e., high power and low power) can be achieved by adjusting the current flowing through UV LEDs. This could be accomplished manually or through an automated circuit.

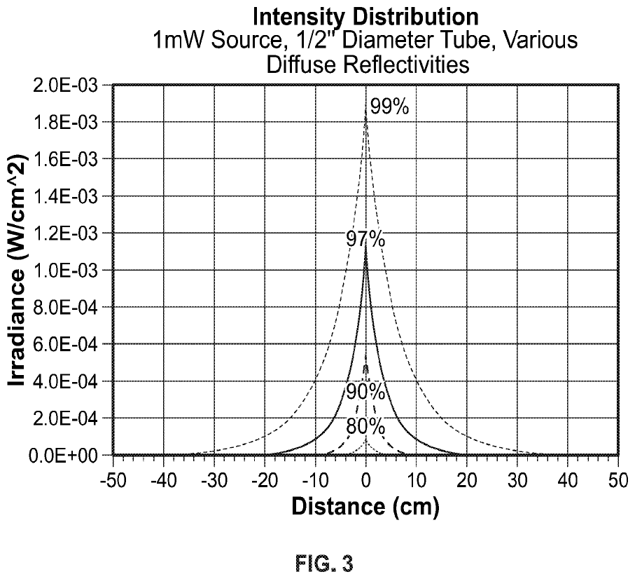

The light distribution of the UV light disinfecting system 1 was modeled using TracePro, a commercially available optical ray tracing software package. FIG. 3 is a graphical illustration depicting the light distribution of a 0.5″ inner diameter tube when a 1 mW output power point source is mounted on the exterior surface of the tube for various diffuse reflectivities on the inner tube wall 10. As shown in FIG. 3, the light extends only a couple centimeters at 80% total diffuse reflectance but tens of centimeters at 99% diffuse reflectance.

It is one objective of the present invention to mitigate the growth of biofilms on the inner walls 10 of the highly reflective tube 2. How far the UV-light must extend along the inner walls 10 of the reflective tube 2 depends on the intensity or irradiance required to prevent a biofilm from growing. This depends on the type of bacteria as well as the wavelength of the UV source. Salters and Piola, in their article “UVC Light for Antifouling”, cite very low power levels at the surface are required, on the order of 1 mW/m² which equates to 100 nW/cm².

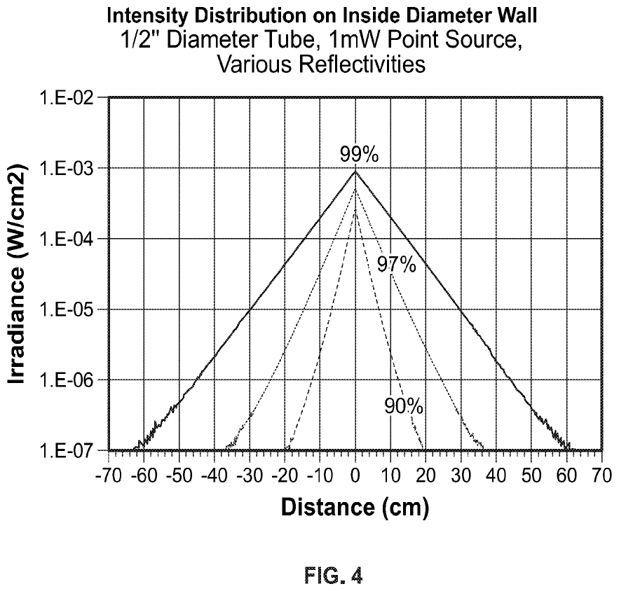

FIG. 4 depicts the same plot as shown in FIG. 3, but on a log scale. FIG. 4 shows that at a 90% diffuse reflectance, a 1 mW point source reaches 20 cm in each direction for a total span of 40 cm before the irradiance drops to 100 nW/cm². At 99% reflectance the total span is 120 cm.

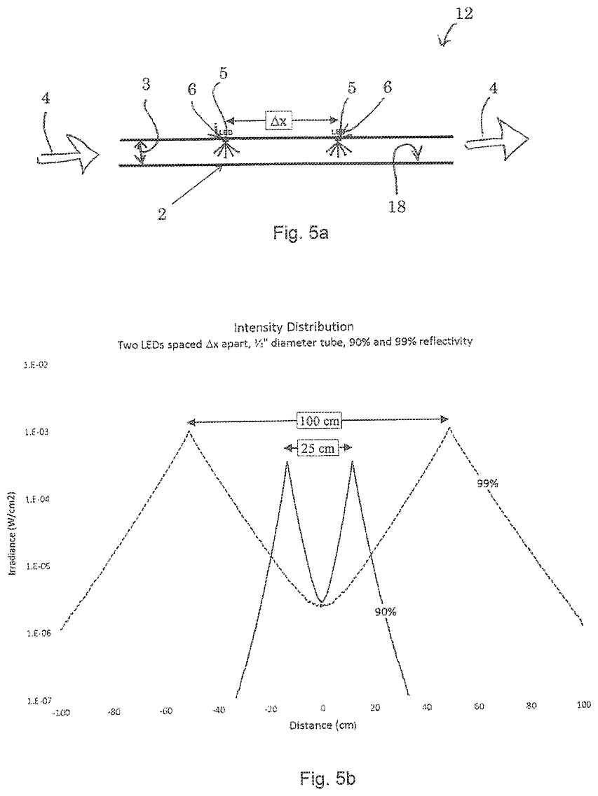

FIG. 5a is a schematic illustration of a side cross-sectional view of a UV disinfecting system in accordance with another embodiment. UV disinfecting system 12 is the same as the UV disinfecting system 1 depicted in FIG. 1 with the exception that two UV-LEDs 5 are attached to the outer surface of the highly reflective tube 2 and such that the UV-LEDs are aligned with the openings 6. It is to be appreciated that more than two UV-LEDs may be mounted on the surface along the length of the tube 2, such as with an array of UV-LEDs. The UV-LEDs 6 are spaced a distance Δx apart from each other. Directional arrows 4 indicates the flow of water or air through the highly reflective tube 2 and the diameter of the reflective tube 2 is indicated by reference numeral 3.

FIG. 5b is a graphical illustration of the light distribution of the UV disinfecting system 12 shown in FIG. 5b for 90% diffuse reflectance walls when the distance between the UV-LEDs 6 is 25 cm and for 99% diffuse reflectance walls when the distance between the UV-LEDs 6 is 100 cm. Equidistant between the UV-LEDs 6 the light irradiance drops to a minimum intensity level, in this case, approximately 2 uW/cm². This minimum intensity level on the surface wall of the highly reflective tube needs to be above the intensity level required to prevent biofilms from growing, which, in this example, is an order of magnitude above the estimated minimum irradiance level of 100 nW/cm² required to prevent the formation of biofilms.

In order to prevent the growth of biofilms on surfaces through the use of UV light, the design of the UV light disinfecting system must ensure that the light emitted from the UV-LED sources reach all surfaces desired to be disinfected. The most efficient method to achieve this objective is through highly diffuse reflector materials. A material with specular reflection will not disperse the UV light rays enough to uniformly distribute the UV light power to all desired surfaces. Thus, the use of a material with specular reflection may create zones of high light intensity and zones of lower light intensity (e.g., “hot” and “cold” spots). Regions of lower light intensity are areas where biofilms may grow.

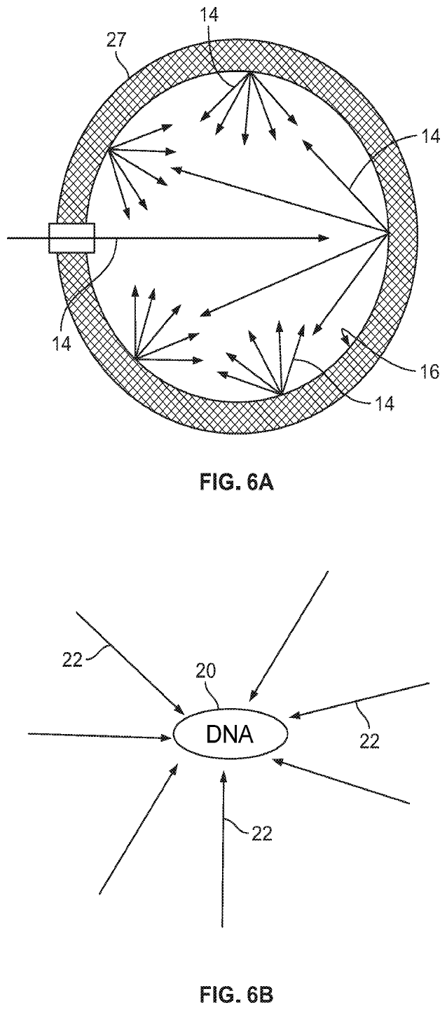

The optics design approach of the present invention is similar to optical integrating spheres which use a highly diffuse reflecting material. The schematic illustration shown in FIG. 6a depicts the scattering of light 14 off the inner walls 16 of a sphere or cylinder 27. In the ideal case of 100% diffuse reflecting walls the same photon flux from all angles exists in every microvolume, thus enabling a uniform fluency rate throughout the volume of the sphere or cylinder 27. Additionally, all surfaces of the inner volume of the sphere or cylinder 27 are also being impinged with light of the same irradiance level and thus no cold spots exist. Such an approach is also beneficial to disinfecting pathogens in a water or fluid medium. FIG. 6b depicts a pathogen 20 where UV light 22 impinges on the pathogen 20 from all angles, which is more effective at inactivating the DNA in the pathogen than impinging the pathogen 20 with UV light from one side only.

In at least one embodiment, the UV light disinfecting system uses highly reflecting materials. For example, the UV light disinfecting system may use a material that has greater than 80% reflectance or greater than 90% reflectance, where the diffuse component of the total reflection is greater than 90% and the specular component is less than 10%. For example, if the total reflection is 90%, the reflection consists of a minimum 81% diffuse reflectance or maximum 9% specular reflectance.

Reflective tubes of diffuse UV reflectivity 80% or greater can be produced through a number of different methods. One exemplary method is to wrap a film having a high diffuse reflectivity in a helical or longitudinal manner to form a helically wrapped tube as discussed in PCT patent application number PCT/US2017/065590 to Donhowe, et al. Another exemplary method of forming a reflective tube is through extrusion. An exemplary embodiment of a polytetrafluoroethylene (PTFE) tube formed via extrusion is described in U.S. Pat. No. 5,620,763 to House, et al.

A third exemplary method of forming optical tubes is through electrospinning. Electrospinning refers to a process for forming mats, tubes, or other shapes by depositing small strings of a polymer on a surface. The production process uses charged electric forces to melt polymer solutions to produce sub-nanometer or nanometer sized fibers. A specific arrangement of the fibers produced can be used to manufacture a highly diffuse reflective material, e.g., 90% or greater. This highly diffuse reflective material can be subsequently wrapped into a tubular shape as described in PCT patent application number PCT/US2017/065590 to Donhowe, et al. Alternatively, the electrospinning process can be used to form tubes directly without subsequent wrapping. U.S. Pat. No. 8,178,030 to Anneaux, et al. describes a process for electrostatic spinning of PTFE to form tubes.

Materials that may be used in the UV light disinfecting system have high reflection coefficients, such as greater than about 80% reflectivity, greater than 90% reflectivity, or greater than about 98% reflectivity. In exemplary embodiments, the material also does not exhibit degradation under UV light radiation. Many polymers degrade under UV light and exhibit yellowing and an increase in absorption. It is also desirable for the highly diffuse reflective material to exhibit low water absorption and hydrophobicity.

A variety of materials are candidates for construction of the UV light disinfecting system. Suitable polymers for use in the reflective tube include, but are not limited to, a fluoropolymer, a polyimide, a polyolefin, a polyester, a polyurethane, a polyvinyl, polymethyl methacrylate, or variations or combinations thereof. Exemplary polymers include, but are not limited to, polyethylene terephthalate (PET), polyethylene naphthalate (PEN), poly ether ether ketone (PEEK), cyclic olefin copolymer (COC), polycarbonate (PC), polyphenylene sulfide (PPS), polyetherimide (PEI), polyamideimide (PAI), polychloroprene, polyvinyl chloride (PVC), polyvinylidene chloride (PVDC), vinylidene chloride-vinyl chloride copolymers, vinyl chloride copolymers, vinylidene fluoride polymers, polyvinylidene fluoride (PVDF), fluorinated ethylene propylene (FEP), perfluoroalkoxy alkane (PFA), or polytetrafluoroethylene (PTFE).

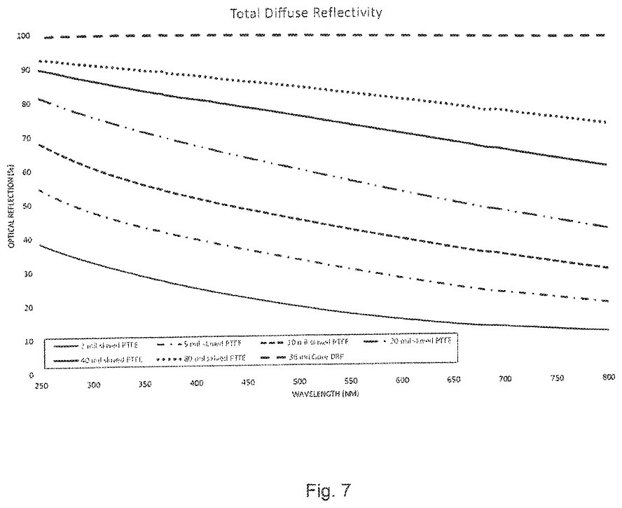

In some embodiments, the polymer is an expanded polytetrafluoroethylene (ePTFE). Expanded polytetrafluoroethylene (ePTFE) is advantageous in that it is hydrophobic, has low water absorption, low optical absorption in the UV light spectrum (such as light having wavelengths between 200 nm and 400 nm), and can be made to have a high diffuse optical reflection coefficient. FIG. 7 is a graphical illustration depicting the reflection coefficient of various forms of ePTFE. As shown in FIG. 7, the commercial product Gore® DRP exhibits 99% total diffuse reflectivity in the ultraviolet (UV) spectrum.

In some embodiments, the reflective tube may include or be formed of an expanded polytetrafluoroethylene (ePTFE) material. In some embodiments, the reflective tube includes a thin metal film. In some embodiments, the reflective tube is aluminum. Aluminum is an exemplary metal that shows higher reflectivity in the UV spectrum compared to other metals.

In some embodiments, the reflective tube is aluminum filled fluoropolymer. In some embodiments, the reflective tube is aluminum filled PET. In some embodiments, the reflective tube is aluminum filled PVC. In some embodiments, the reflective tube is aluminum filled PVDC. In some embodiments, the reflective tube is aluminum filled PC.

In some embodiments, the reflective tube is aluminum filled ePTFE.

In some embodiments, the reflective tube wall includes a dielectric stack. In some embodiments, the reflective tube includes a porous layer. In some embodiments, the reflective tube may be a combination of different layers. In one exemplary embodiment, the reflective tube is an ePTFE inner layer surrounded by an aluminum foil layer.

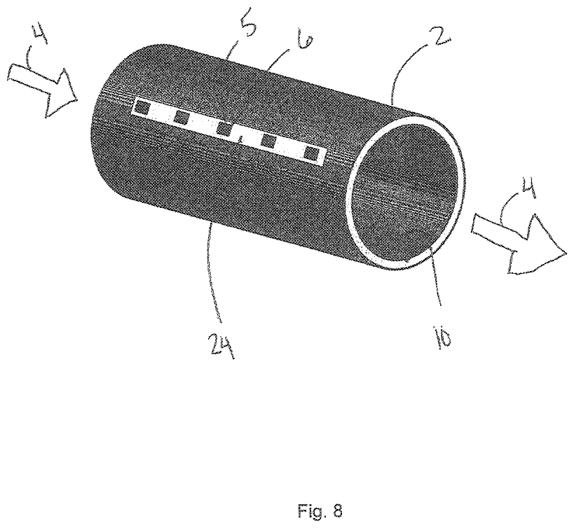

In some embodiments of the present disclosure, construction of a UV light disinfecting system includes mounting UV sources such as UV-LEDs (light emitting diodes) on the external surface of the reflective tube. A schematic illustration of an exemplary UV light disinfecting system is depicted in FIG. 8. As shown, the UV light disinfecting system includes a reflective tube 2 having a reflective inner surface 10, and integrated UV LED array positioned on the exterior surface of the reflective tube 2. Since the walls of the tube 2 have a high reflection coefficient, an opening is required to enable the light emitted by the UV-LED to enter the inside of the tube 2 and impinge on a wall opposing the UV-LED. Openings in the reflective tube 2 can be formed by cutting openings in the tube 2 through various processes such as laser cutting, die punching, or drilling. Alternatively, openings can be formed during a wrapping process, such as is described in PCT patent application PCT/US2017/065590 to Donhowe, et al.

The UV-LEDs used in the UV light disinfecting system may be mounted on a strip which can include the circuitry necessary to power the UV-LEDs. The strip may be a flexible printed circuit board or, alternative, the strip may be rigid. In addition, the strip may include a heat sink to enable the UV-LEDs to cool off. The UV-LED strip may include one or multiple LEDs, such as in the form of an array. The distance (ΔX) between the UV-LEDs can vary anywhere from centimeters to a meter. The UV-LED strip may be mounted to the reflective tube with adhesives or other securing methods such as wrapping another material around the reflective tube and UV-LED strip or array.

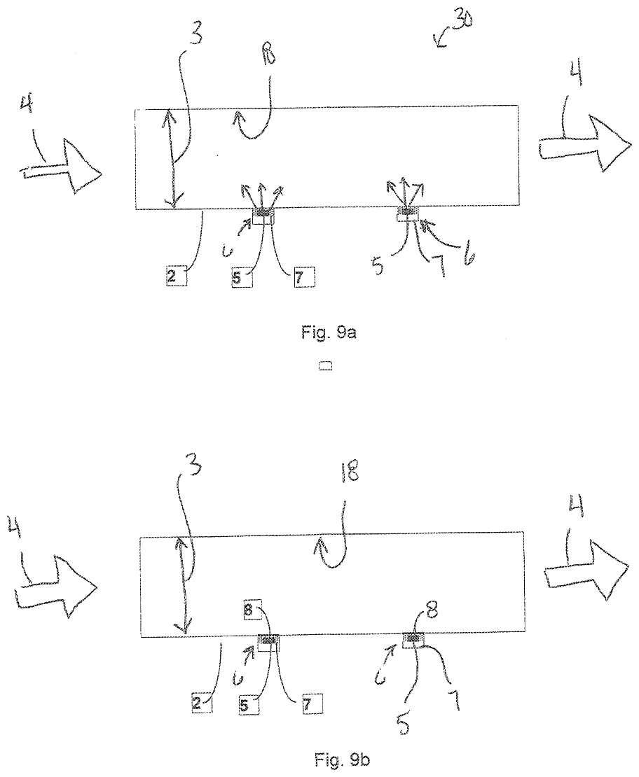

The pitch or spacing between the UV-LEDs on the UV-LED strip or array, and the corresponding openings in the wall of the reflective tube to which the UV-LEDs align thereto, are pre-determined and are based on the optics design required to maintain a minimum irradiance level throughout the highly reflective tube, an example shown in FIG. 5a. The construction of a UV light disinfecting system requires a water leak tight design with some applications requiring no water leaks at pressures up to 200 psi. A potential leak point is the openings cut in the side wall where the UV-LEDs are positioned. In one embodiment of a UV light disinfecting system 30 depicted generally in FIG. 9a, the opening in the reflective tube 2 is filled with an encapsulant 7. The encapsulant 7 may provide water resistance or other environmental protection to the UV-LEDs 5. In some embodiments, the encapsulant 7 adheres to the side walls of the opening as well as to the UV-LEDs 5. The encapsulant 7 can be formed of a solvent-based material or a resin. Exemplary encapsulants include fluorinated ethylene propylene (FEP), perfluoroalkoxy alkane (PFA), a terpolymer of tetrafluoroethylene, hexafluoropropylene, and vinylidene fluoride (THV), a copolymer of FEP and polyethylene (EFEP), and silicone. Directional arrows 4 indicates the flow of water or air through the highly reflective tube 2 and the diameter of the reflective tube 2 is indicated by reference numeral 3.

An alternative embodiment of a UV light disinfecting system is shown in FIG. 9b. The UV light disinfecting system 40 includes a reflective tube 2, UV-LEDs 5, an encapsulant 7, and a transparent film button 8. The transparent film button 8 has the shape and size of the opening and is placed between the encapsulant 7 and UV-LED 5 and the tube opening. Directional arrows 4 indicates the flow of water or air through the highly reflective tube 2 and the diameter of the reflective tube 2 is indicated by reference numeral 3.

In practice, the reflective tube 2 with openings 6 is placed over a temporary mandrel, then the button film 8 and the encapsulant 7 are added into the opening. The UV-LED strip or array is then aligned and placed over the openings 6. In exemplary embodiments, the encapsulant 7 fills the opening 6 such that no air pockets are present. In other embodiments, the encapsulant 7 adheres to the transparent button film 8, the opening 6 in the surface of the tube 2, and the UV-LED 5. The button film 8 covers the opening in the reflective tube 2 and is positioned on the interior surface 18 of the tube 2.

An alternative method to prevent water leaks is to wrap a film around the highly reflective tube, including the openings therein. In some embodiments, the film is optically transparent. The UV-LED strip is then aligned with the openings in the surface of the reflective tube 2 and pressed against the surface of the reflective tube 2 such that the UV-LEDs 5 push against the transparent film. In some embodiments, the transparent film is elastic or has some elasticity in order to conform to the UV-LED structure. Exemplary films include polytetrafluoroethylene (PTFE), fluorinated ethylene propylene (FEP), perfluoroalkoxy alkane (PFA), a terpolymer of tetrafluoroethylene, hexafluoropropylene, and vinylidene fluoride (THV), a copolymer of FEP and polyethylene (EFEP).

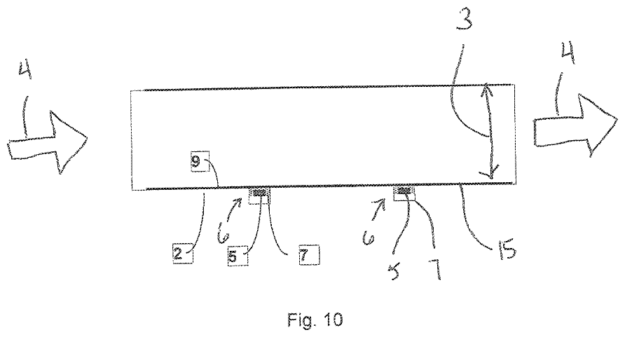

A further embodiment of a UV light disinfecting system is depicted in FIG. 10. An optically transparent inner tube 9 is used to create a leak tight tube that is also a barrier layer to prevent fluids from penetrating the walls of the reflective 2 tube and compromising the UV-LEDs 5 and associated electronics. An encapsulant 7 may be utilized to provide water resistance or other environmental protection to the UV-LEDs 5. The transparent inner tube 9 may have a transmission coefficient of at least 80% to UV light or greater than 90%. As discussed herein, the reflective tube 2 can be constructed by extrusion or wrapping of films around a mandrel. Fluoroploymer materials such as fluorinated ethylene propylene (FEP), hexafluoropropylene, and vinylidene fluoride (THV), a copolymer of FEP and polyethylene (EFEP), perfluoroalkoxy alkane (PFA), or polytetrafluorethylene (PTFE) can be used to form the reflective tube 2. In exemplary embodiments, the reflective tube 2 is constructed so as to maintain a minimum internal water pressure of 100 psi or, in some embodiments, 200 psi. Directional arrows 4 indicates the flow of water or air through the highly reflective tube 2 and the diameter of the reflective tube 2 is indicated by reference numeral 3.

Surrounding the transparent inner tube 9 is the reflective tube 2 which contains the pre-determined openings 6 in which the spacing between the openings 6 matches the spacing between the UV-LEDs 5. The reflective tube 2 with openings 6 may be constructed using methods described previously. The reflective tube 2 can be attached to the inner transparent tube 9 with adhesives such as fluorinated ethylene propylene (FEP), perfluoroalkoxy alkane (PFA), a terpolymer of tetrafluoroethylene, hexafluoropropylene, and vinylidene fluoride (THV), a copolymer of FEP and polyethylene (EFEP), and silicone. An alternative method is to join the two tubes 2, 9 via a heat set process. In such a process, the two tubes 2, 9 are aligned over a mandrel which is then heated to the temperature at which the outer reflective tube 2 shrinks to make a tight fit to the inner tube 9, or to the temperature at which at least one of the tubes 2, 9 begins to soften. An alternative method is to slide the outer reflective tube 2 over the inner transmissive tube 9 but not use an adhesive between the two tubes 2, 9. A UV-LED array 15 can then be attached to the outer reflective tube 2 using any of the attachment methods described previously.

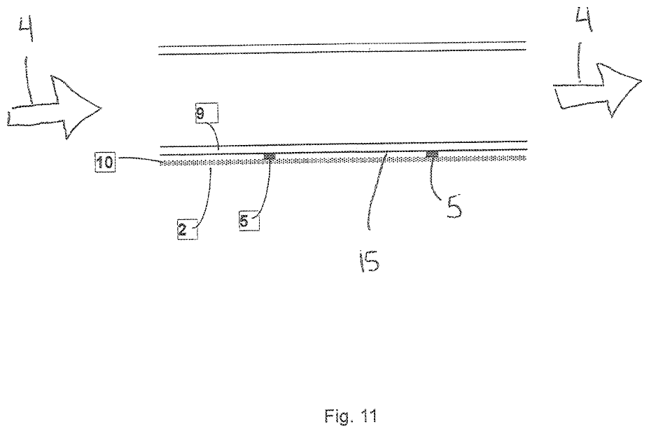

A further embodiment of the UV light disinfecting is shown in FIG. 11. In this embodiment, a UV-LED array 15 is attached to the outside of the transparent tube 9. The UV-LED array 15 includes a substrate 10 to support and provide power to the UV-LEDs 5. An exemplary embodiment of the substrate is a flexible printed circuit board. The substrate 10 may also include a metal bar (not depicted) to distribute heat away from the UV-LEDs 5. The outer reflective tube 2 is then slid over the combined inner transparent tube 9 plus UV-LED array 15. In this embodiment, openings in the reflective tube are not required. Directional arrows 4 indicates the flow of water or air through the highly reflective tube 2.

In an alternative embodiment, the UV-LED array 15 may be mounted inside either a reflective tube 2 or inside the combination of a transparent inner tube 9 plus outer reflective tube 2. In this embodiment, the UV-LEDs 5 and UV-LED array 15 are in contact with the water or air flowing through the tube 2. The UV-LED array 15 can be attached to the inner wall 18 of the reflective tube 2 with an adhesive. The UV-LED strip can be free floating (e.g., not attached to the inner wall of the tube 2), but may need to be secured upstream or downstream from the fluid flow to prevent the UV-LED array 15 from moving. For example, the UV-LED array 15 may be temporarily inserted inside a wall of a pipe to remove biofilms that have started to grow.

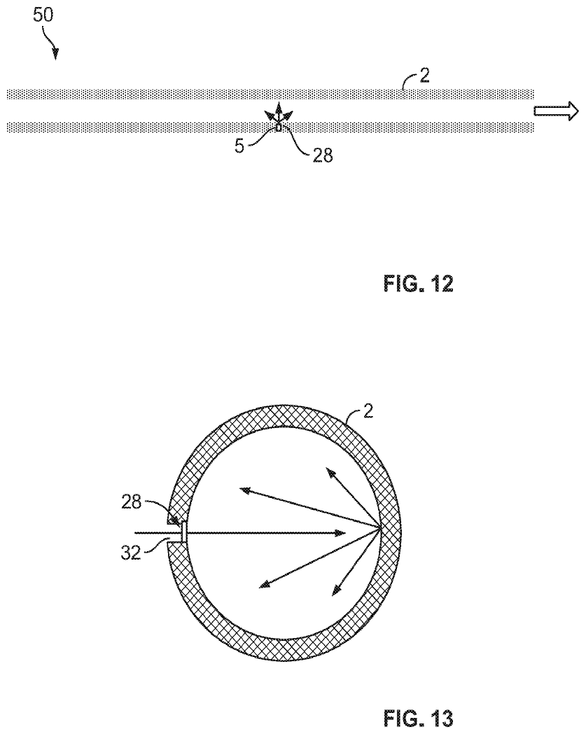

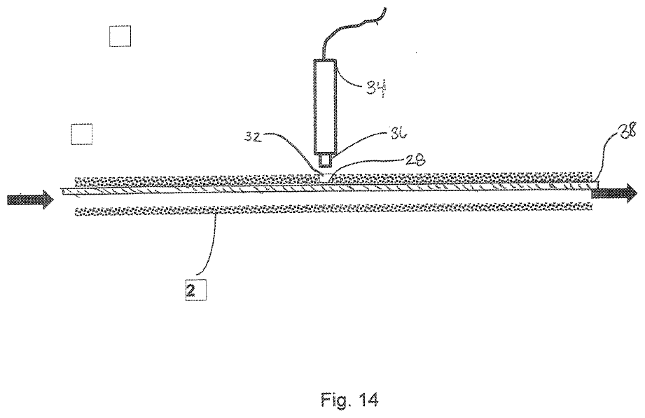

Another alternative embodiment of a UV light disinfecting system is shown in FIG. 12. The UV light disinfecting system 50 includes a reflective tube 2, a UV-LED 5 and a transparent window 28 within the reflective tube 2. Although FIG. 12 depicts one transparent window 28, any number of transparent windows 28 may be incorporated into the reflective tube 2. The transparent windows 28 may be formed in a variety of shapes including circles, ovals, triangles, squares, rectangles, diamonds and other similar shapes. The size of the transparent windows 28 may also vary so long as the transparent windows 28 are sufficient to allow light from a UV-LED 5 to pass through and impinge upon the wall opposing the UV-LED 5, as depicted in FIG. 14.

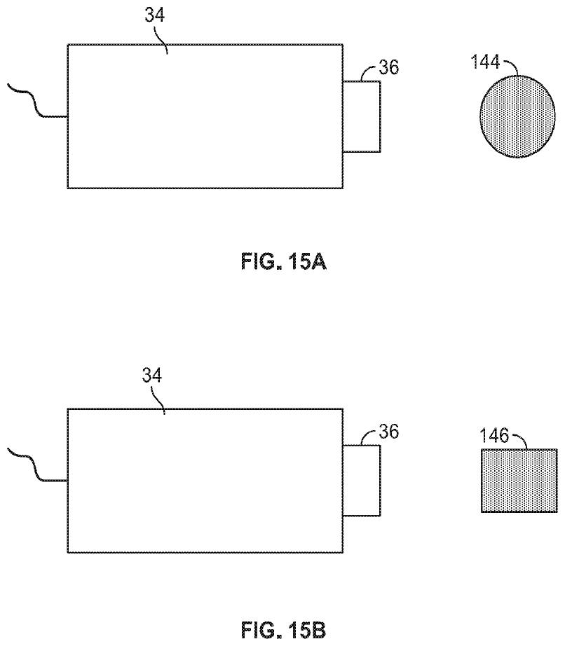

The transparent windows 28 eliminate the cutting of openings into the reflective tube 2 during the construction process of the system 50, thus preventing pathogens or substances within the reflective tube 2 from escaping. The transparent windows 28 may be formed within the walls of the existing reflective tube 2. For example, in an exemplary embodiment, the translucency of the transparent window 28 is obtained by a process in which regions of the polymer material—i.e., ePTFE—of the reflective tube 2 are selectively compressed to eliminate air therein. Such a process, in some embodiments, comprises applying pressure and heat to the reflective tube 2 using a heating/forming tool 34, depicted in FIG. 14. The heating tool 34 includes a compression form 36 that contacts the reflective tube 2 to apply heat and pressure to form the transparent windows. In some embodiments the compression form 36 has a circular cross-section 144, as depicted in FIG. 15a or a rectangular cross-section 146, as depicted in FIG. 15b. In other embodiments, the compression form 36 may have any other shape or size.

In some embodiments, the heating/forming tool 34 is used in conjunction with a support member 38 on the inside of the reflective tube 2 to simultaneously heat and compress the material of the reflective tube 2 at selected locations where transparent windows 28 are to be located, as depicted in FIG. 14. For example, in exemplary embodiments, the forming tool and the support member apply pressure in the range of 1000 psi to 25000 psi to the selected location of the reflective tube 2. In other embodiments, pressure applied to the selected location of the reflective tube 2 is in the range of 5000 psi to 25000 psi. In other embodiments, pressure applied to the selected location of the reflective tube 2 is in the range of 9000 psi to 25000 psi. In other embodiments, pressure applied to the selected location of the reflective tube 2 is in the range of 10000 psi to 25000 psi. In other embodiments, pressure applied to the selected location of the reflective tube 2 is in the range of 12500 psi to 25000 psi. In other embodiments, pressure applied to the selected location of the reflective tube 2 is in the range of 15000 psi to 25000 psi. In other embodiments, pressure applied to the selected location of the reflective tube 2 is in the range of 17500 psi to 25000 psi. In other embodiments, pressure applied to the selected location of the reflective tube 2 is in the range of 20000 psi to 25000 psi. In other embodiments, pressure applied to the selected location of the reflective tube 2 is in the range of 22500 psi to 25000 psi.

In some embodiments, pressure applied to the selected location of the reflective tube 2 is in the range of 1000 psi to 22500 psi. In other embodiments, pressure applied to the selected location of the reflective tube 2 is in the range of 1000 psi to 18000 psi. In other embodiments, pressure applied to the selected location of the reflective tube 2 is in the range of 1000 psi to 15000 psi. In other embodiments, pressure applied to the selected location of the reflective tube 2 is in the range of 1000 psi to 10000 psi. In other embodiments, pressure applied to the selected location of the reflective tube 2 is in the range of 1000 psi to 7500 psi. In other embodiments, pressure applied to the selected location of the reflective tube 2 is in the range of 1000 psi to 5000 psi. In other embodiments, pressure applied to the selected location of the reflective tube 2 is in the range of 1000 psi to 2500 psi.

In some embodiments, the pressure applied to the selected location of the reflective tube 2 is in the range of 6000 psi to 12500 psi. In other embodiments, pressure applied to the selected location of the reflective tube 2 is in the range of 7000 psi to 9000 psi. In other embodiments, pressure applied to the selected location of the reflective tube 2 is in the range of 8500 psi to 13000 psi. In other embodiments, pressure applied to the selected location of the reflective tube 2 is in the range of 12500 psi to 14000 psi. In other embodiments, pressure applied to the selected location of the reflective tube 2 is in the range of 18000 psi to 22000 psi. In other embodiments, pressure applied to the selected location of the reflective tube 2 is in the range of 5000 psi to 15000 psi.

In some embodiments, the forming tool 34 applies heat to the reflective tube 2 in the range of 100° C. to 300° C. In other embodiments, the forming tool 34 applies heat to the reflective tube 2 in the range of 100° C. to 250° C. In other embodiments, the forming tool 34 applies heat to the reflective tube 2 in the range of 100° C. to 200° C. In other embodiments, the forming tool 34 applies heat to the reflective tube 2 in the range of 100° C. to 150° C.

In some embodiments, the forming tool 34 applies heat to the reflective tube 2 in the range of 150° C. to 300° C. In other embodiments, the forming tool 34 applies heat to the reflective tube 2 in the range of 200° C. to 300° C. In other embodiments, the forming tool 34 applies heat to the reflective tube 2 in the range of 250° C. to 300° C.

In some embodiments, the forming tool 34 applies heat to the reflective tube 2 in the range of 150° C. to 250° C. In other embodiments, the forming tool 34 applies heat to the reflective tube 2 in the range of 200° C. to 250° C. In other embodiments, the forming tool 34 applies heat to the reflective tube 2 in the range of 150° C. to 200° C.

This heated compression of the selected locations of the reflective tube 2 collapses the air within the material of the reflective tube 2, forming areas of high transparency for UV light, i.e., the transparent windows 28. Table 1 below describes exemplary heating and pressure conditions used to achieve different UV transparencies within an ePTFE reflective tube 2.

TABLE 1
Exemplary Heat and Pressure conditions
to achieve UV transparency at 265 nm

| Pressure | | | |
|---|---|---|---|
| Temperature | 5000 psi | 10000 psi | 15000 psi |
| 100 C. | 74% | 82% | 88% |
| 200 C. | 78% | 84% | 89% |
| 250 C. | 78% | 85% | 90% |

In some embodiments, filling resins can also be applied to the material of the reflective tube 2, along with heat and pressure to form the transparent windows 28. In an exemplary embodiment, the material of the reflective tube 2 to be filled comprises ePTFE. Exemplary filling resins include, but are not limited to, any thermoplastic or polymer based solution that is used to fill voids within the material of the reflective tube to provide transparency to the material. Filling resins, in some embodiments, include fluorinated ethylene (FEP), perfluoroalkoxy alkane (PFA), THV, EFEP, a copolymer of ethylene, PATT, PZM4, silicones, fluorosilicones, other UV non-light scattering stable filling resins, or combinations thereof.

In some embodiments, the filling resin comprises polytetrafluoroethylene (PTFE).

Table 2 below describes typical heating and pressure conditions which are used with an FEP resin to achieve different UV transparencies within an ePTFE reflective tube 2.

TABLE 2
Typical Heat and Pressure conditions with an
FEP resin to achieve UV transparency at 265 nm

| Pressure | | | |
|---|---|---|---|
| Temperature | 5000 psi | 10000 psi | 15000 psi |
| 100 C. | 82% | 86% | 90% |
| 200 C. | 82% | 88% | 90% |
| 250 C. | 82% | 88% | 91% |

Alternately, in some embodiments, polymer-based filling resins can be applied to the material of the reflective tube 2 without heat and pressure to form the transparent windows 28. In these embodiments, filling resin content and the material of the reflective tube 2 are optimized to achieve transparent windows by processes described in U.S. Pat. Nos. 6,451,396 and 6,737,158 to W. L. Gore.

In exemplary embodiments, the transparent windows 28 have a very low optical absorption (e.g., less than 10%, less than 5%, or less than 1%) so that a very high percentage of the light is transmitted through the transparent windows 28. In some embodiments, the transparent windows 28 exhibit a transparency of 70% or greater, 75% or greater, 80% or greater, 90% or greater or 95% or greater for UV light having wavelengths between 100 nm and 400 nm. In other embodiments, the transparent windows 28 exhibit a transparency of 70% to 100% for UV light wavelengths between 100 nm and 400 nm. In other embodiments, the transparent windows 28 exhibit a transparency of 80% to 100% for UV light wavelengths between 100 nm and 400 nm. In other embodiments, the transparent windows 28 exhibit a transparency of 90% to 100% for UV light wavelengths between 100 nm and 400 nm. In other embodiments, the transparent windows 28 exhibit a transparency of 95% to 100% for UV light wavelengths between 100 nm and 400 nm.

In some embodiments, applying pressure and heat to the reflective tube 2 condenses the highly reflective material of the reflective tube 2 such that a chamber 32 is created within the reflective tube 2 adjacent to the transparent window 28, as depicted in FIGS. 13-14. Exemplary transparent windows 28, in some embodiments, have a thickness of 5 microns to 250 microns. In other embodiments, the transparent windows 28 have a thickness of 50 microns to 250 microns. In other embodiments, the transparent windows 28 have a thickness of 75 microns to 250 microns. In other embodiments, the transparent windows 28 have a thickness of 125 microns to 250 microns. In other embodiments, the transparent windows 28 have a thickness of 175 microns to 250 microns. In other embodiments, the transparent windows 28 have a thickness of 225 microns to 250 microns.

In some embodiments, the transparent windows 28 have a thickness of 20 microns to 250 microns. In other embodiments, the transparent windows 28 have a thickness of 75 microns to 250 microns. In other embodiments, the transparent windows 28 have a thickness of 100 microns to 250 microns. In other embodiments, the transparent windows 28 have a thickness of 150 microns to 250 microns. In other embodiments, the transparent windows 28 have a thickness of 175 microns to 250 microns. In other embodiments, the transparent windows 28 have a thickness of 200 microns to 250 microns.

In other embodiments, the transparent windows 28 have a thickness of 50 microns to 200 microns. In other embodiments, the transparent windows 28 have a thickness of 80 microns to 160 microns. In other embodiments, the transparent windows 28 have a thickness of 100 microns to 200 microns. In other embodiments, the transparent windows 28 have a thickness of 150 microns to 175 microns.

In some embodiments, the UV-LED 5 may then be positioned within the chamber 32 or mounted on the external surface of the reflective tube 2 such that the light emitted by the UV-LED 5 passes through the transparent window 28 and into the reflective tube 2 to impinge on a wall opposing the UV-LED 5.

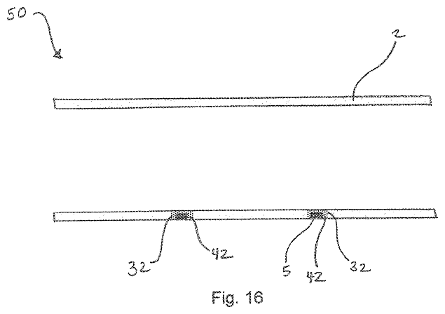

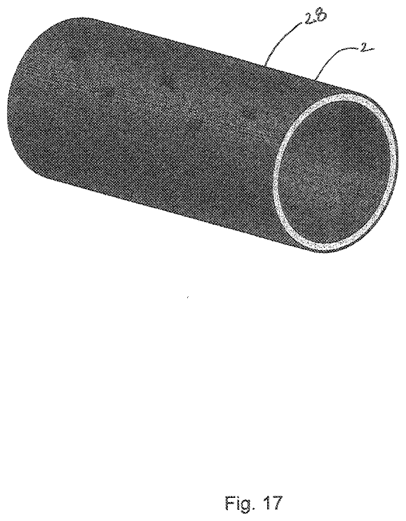

In one embodiment of a UV light disinfecting system 50 depicted generally in FIG. 16, the chambers 32 are filled with an adhesive 42. The adhesive 42 may provide water resistance or other environmental protection to the UV-LEDs 5. In some embodiments, the adhesive 42 adheres to the side walls of the opening as well as to the UV-LEDs 5. The adhesive 42 can be formed of a solvent-based material or a resin. Exemplary adhesives include fluorinated ethylene propylene (FEP), perfluoroalkoxy alkane (PFA), a terpolymer of tetrafluoroethylene, hexafluoropropylene, and vinylidene fluoride (THV), a copolymer of FEP and polyethylene (EFEP), and silicone.

The pattern of the transparent windows 28 can be any pattern desired to achieve optimal power requirements within the reflective tube 2. For example, in some embodiments, the transparent windows 28 are spaced at regular or irregular intervals and uniformly or non-uniformly distributed along a length of the reflective tube 2. In other embodiments, the transparent windows 28 are arranged in a parallel configuration of in a staggered configuration, depicted in FIG. 17.

In some aspects of the present disclosure, multiple UV-LEDs are arranged as an array of UV-LEDs and conductors, with one or more of the UV-LEDs and conductors connected to and/or attached to others in the array by, for example, electrical connections. The UV-LED array may be regular or non-regular, meaning the UV-LEDs and conductors may be uniformly distributed or non-uniformly distributed. An example array may correspond to a ribbon cable, flexible circuit, or flat flexible cable having UV-LEDs attached along various positions of the ribbon cable, flexible circuit, or flat flexible cable. In embodiments where a UV-LED array is used, the transparent windows 28 may be positioned to correspond to the UV-LED array so as to optimize the transmittal of the UV light to the interior of the reflective tube 2.

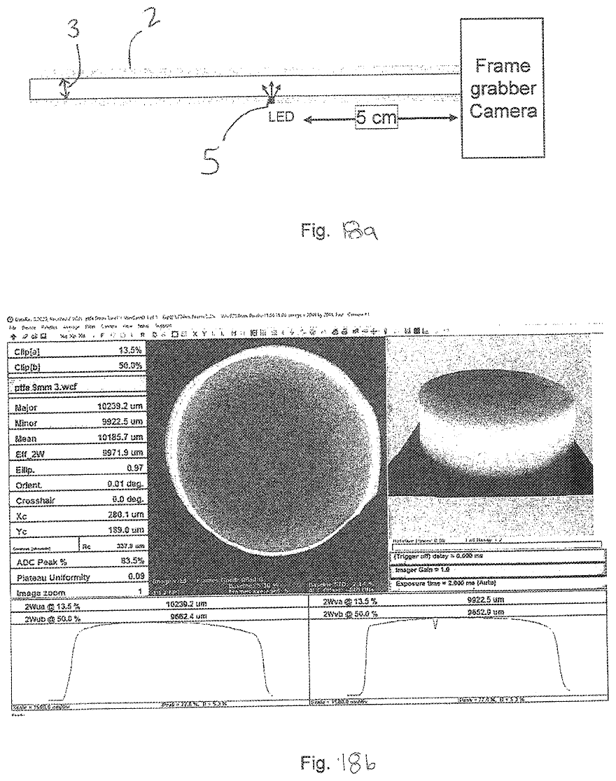

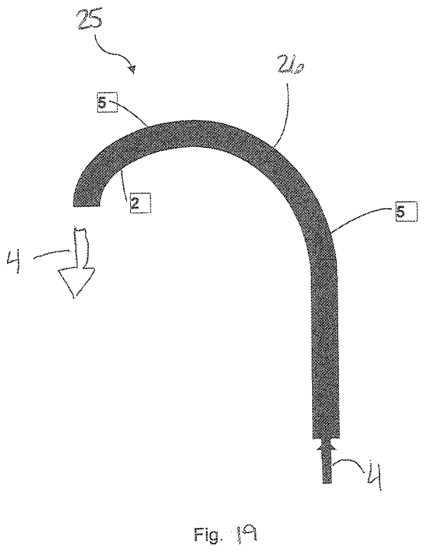

It is an objective to provide a UV light disinfecting system that includes at least a reflective tube 2 and at least one UV-LED that emits UV light into the interior volume of the tube 2 and which disinfects pathogens and prevents biofilm growth by uniformly illuminating the inner volume of the tube 2 with constant UV light. To test that objective, a 97% diffuse reflective tube was constructed, an opening was cut in the surface of the tube 2, and a UV-LED was inserted in the opening, as shown in FIG. 18a. The tube 2 was cut 5 cm from the UV-LED and butted up against the pixels of a frame grabber camera array. FIG. 18b shows the results which illustrate a uniform “top hat” distribution profile which confirms a uniform intensity distribution across the diameter 3 of the tube 2.

It is also an objective to provide an article that is tubular and flexible such that it can fit inside plumbing fixtures. A non-limiting example is shown in FIG. 19, which depicts a UV disinfecting gooseneck faucet 25. The gooseneck outer tube 26 houses a UV light disinfecting system inside. The UV light disinfecting system has a reflective wall 2 with UV-LEDs 5 placed periodically along the tube 2. It is a further objective to provide a plumbing fixture that prevents the growth of biofilms by placing UV-LEDs periodically along the tubing such that the light irradiance is a minimum of 100 nW/cm2 on the tubing wall surface. The number of LEDs required will depend on the resistivity of the disinfecting tube walls as shown in FIGS. 4 and 5, but at least one UV-LED is necessary with no upper limit on the maximum number of UV-LEDs or UV-LED arrays.

In operation, the UV-LEDs may be constantly turned on to prevent the growth of biofilms on the surface walls. Alternatively, the UV-LEDs may be pulsed on periodically. The UV-LEDs used in the construction of the UV light disinfecting system may be low power UV-LEDs, for example on the order of 1 mW output power, and only used to prevent biofilm growth. Alternatively, the UV-LEDs used may be high power UV-LEDs, for example 10 or 100 mW output power, and driven at these high powers to disinfect pathogens in the fluid when the fluid is flowing; then driven at lower current levels to prevent biofilm growth when the fluid is not flowing. Driving the UV-LEDs at lower current levels will conserve energy and UV-LED lifetime. The fluid is typically water but may be other fluids where it is desired to disinfect pathogens. The UV-LEDs emit a wavelength in the UV light range which is less than 400 nm, or in the 250 nm to 280 nm range.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A UV light disinfecting system comprising:
a flexible, reflective tube having an outer wall defining an open interior region,
wherein the flexible, reflective tube comprises:
i) an inner reflective surface; and
ii) at least one feature in the outer wall of the flexible, reflective tube configured to guide UV light into the open interior region;
wherein the flexible, reflective tube has a total reflectivity of more than 90% and a diffuse UV reflectivity of more than 80%;
at least one UV-LED coupled to the flexible, reflective tube and aligned with the at least one feature; and
an electronic set up to power the at least one UV-LED;
wherein the at least one UV-LED is configured to scatter UV light emitted therefrom along a length of the flexible, reflective tube to homogeneously illuminate the open interior region.
wherein the at least one UV-LED is configured to scatter UV light emitted therefrom

2. The UV light disinfecting system of claim 1, wherein the at least one feature comprises at least one opening.

3. The UV light disinfecting system of claim 1, wherein the at least one UV-LED is positioned external to the flexible, reflective tube.

4. The UV light disinfecting system of claim 2, wherein the at least one opening comprises a plurality of openings spaced a distance from each other, and
wherein each of the plurality of openings corresponds to one of the at least one UV-LED.

5. The UV light disinfecting system of claim 2, further comprising an encapsulant in the at least one opening.

6. (canceled)

7. The UV light disinfecting system of claim 1, further comprising an optically transparent inner tube positioned within the open interior region of the flexible, reflective tube.

8. (canceled)

9. The UV light disinfecting system of claim 1,
wherein the at least one feature comprises at least one transparent window in the outer wall of the flexible, reflective tube, and
wherein the at least one transparent window is configured to allow UV light emitted from the at least one UV-LED to pass therethrough from the at least one UV-LED to the open interior region.

10. The UV light disinfecting system of claim 9, wherein at least one transparent window comprises a plurality of transparent windows spaced a distance from each other, and
wherein each of the plurality of transparent windows corresponds to one of the at least one UV-LED.

11. (canceled)

12. The UV light disinfecting system of claim 9, wherein the at least one transparent window has a transparency of 70% to 100% in for UV light wavelengths in a range of 100 nm to 400 nm.

13. (canceled)

14. (canceled)

15. (canceled)

16. (canceled)

17. A method to disinfect liquid flow and to mitigate biofilm formation within plumbing fixtures using the UV light disinfecting system according to claim 1,
wherein the UV light disinfecting system is inserted inside of a plumbing fixture, and
wherein the electronic set up is switched between an active mode with a high UV energy fluency rate when media is flowing through the flexible, reflective tube and a passive mode with a low UV energy fluency rate when no media is flowing.

18. A method comprising:
providing a flexible, reflective tube having an outer wall defining an open interior region;
forming at least one feature in the outer wall,
wherein the at least one feature is configured to guide UV light into the open interior region;
positioning a UV-LED on the flexible, reflective tube such that the UV-LED is aligned with the at least one feature so as to form a UV light disinfecting system.

19. The method of claim 18, wherein the at least one feature comprises at least one opening or at least one transparent window.

20. The method of claim 18, further comprising applying an encapsulant to the at least one feature,
wherein the encapsulant at least partially covers the UV-LED.

21. (canceled)

22. The method of claim 18, further comprising:
providing a transparent inner tube,
wherein the transparent inner tube comprises an outer wall defining an open interior region; and
positioning the UV-LED on an outside of the transparent inner tube such that the UV-LED is located between the flexible, reflective tube and the transparent inner tube to illuminate the open interior region through the transparent inner tube.

23. (canceled)

24. (canceled)

25. (canceled)

26. (canceled)

27. The method of claim 23, further comprising:
positioning the UV-LED within a chamber formed adjacent to the at least one transparent window; and
filling the chamber with an adhesive to encase the UV-LED within the chamber.
